(19) United States
(12) Patent Application Publication
(10) Pub. No.: US 2013/0255694 A1
(43) Pub. Date: Oct. 3, 2013

(54) PERCUTANEOUS DILATATIONAL DEVICE

(71) Applicants: Richard L. Arlow, Cleveland, OH (US); Zachary W. Bloom, Cleveland, OH (US); Brian Hewko, Bay Village, OH (US)

(72) Inventors: Richard L. Arlow, Cleveland, OH (US); Zachary W. Bloom, Cleveland, OH (US); Brian Hewko, Bay Village, OH (US)

(73) Assignee: LIFESERVE INNOVATIONS LLC, Cleveland, OH (US)

(21) Appl. No.: 13/432,147

(22) Filed: Mar. 28, 2012

(51) Int. Cl. A61M 16/00

(52) U.S. Cl. 128/207.29

(57) ABSTRACT

The present invention relates to a medical device for performing surgery in the body using a percutaneous dilatational procedure, one embodiment of which is a percutaneous dilatational tracheostomy (PDT) device having a dilator with a non-uniform surface profile that is optimized to the relevant anatomy in order to reduce, uniformly distribute, and ensure uniform, or constant temporal derivative of, dilatational force throughout dilation. This serves to reduce trauma to the patient, specifically producing a reduction of anterior tracheal wall compression, posterior wall laceration/perforation, and tracheal ring fracture that may subsequently lead to subglottic suprastomal stenosis.

### STATEMENT OF GOVERNMENT INTEREST

This invention was made with no government support.

### FIELD OF THE INVENTION

The present invention relates to medical devices, specifically to improved devices for performing surgery in the body using a percutaneous dilatational procedure.

### BACKGROUND OF THE INVENTION

Surgical procedures are increasingly being performed in a minimally invasive manner, in which tools are inserted through small openings or “-otomies” in the surface anatomy of the body to allow for surgical interventions. This method reduces the amount of harm to patients, decreases recovery time, and may also increase safety and reduce costs associated with the procedure as compared to an open surgical procedure. Percutaneous dilatational tracheostomy (PDT) is one such minimally invasive procedure that has gained popularity to create a tracheostomy, an opening into the airway of a patient, within which to place a breathing tube to facilitate respiration. Among several additional percutaneous tracheostomy techniques described in the prior art, the practice of PDT, which uses gradually tapered dilators to form a tracheostomy, has become the most popularly used technique due to an attractive level of simplicity and relative effectiveness as demonstrated in the literature.

The PDT technique was developed by the physician inventor Dr. Pasquelle Ciaglia and was commercialized as a product now called the Ciaglia Basic Percutaneous Tracheostomy kit (CBP) sold by Cook, Inc. This technique used multiple sequentially sized straight dilators that would dilate the trachea in a stepwise manner. Yet, this method was time consuming and proved difficult for use among the general population of physicians performing tracheostomy.

Thereafter, Ciaglia improved his technique by developing a curved continuously tapered dilator that could be inserted into the body thereby creating the tracheostomy with the use of a single dilator, known commonly as the Ciaglia Blue Rhino (CBR) also sold by Cook, Inc (USPTO U.S. Pat. No. 6,637,435). By integrating the procedural components, the procedure could now be performed faster, with greater ease, and with reduced risks of causing injury to tissues of the trachea, specifically to the posterior wall of the trachea due to the dilators curved orientation. (Byhahn, Wilke, Halbig, Lischke, & Westphal, 2000)

As a result of the invention of the CBR dilator, the single dilator technique has become widely accepted as the gold standard for PDT. While generally more safe and efficacious than CBP, many studies have consistently shown an increase in one specific type of complication known as tracheal ring fractures that occur more commonly in the one-step dilation CBR technique in comparison to the CBP. (Beiderlinden, Karl Walz, Sander, Groeben, & Peters, 2002; Byhahn et al., 2000; S M Edwards, 2001; Thomas, Subramani, & Mitra, 2003) Specifically, different studies have noticed a 2× (CBR: 40.9%, CBP: 20.2%) (Beiderlinden et al., 2002) to 22.25× (S M Edwards, 2001) increase in tracheal ring fracture and disruption respectively with CBR as compared to CBP.

Critical comparison of CBP and CBR techniques seem to show a non-uniform dilatational force throughout dilation even though there is a uniform taper in the CBR with a continuous surface profile. (Byhahn et al., 2000) The curved nature of the CBR results in the presence of a global force maximum approximately half-way along the length of dilation where tracheal ring fracture is commonly observed. (Byhahn et al., 2000) (Ho, Kapila, & Colquhoun-Flannery, 2005) The occurrence of a non-uniform dilatational force with CBR is thought to be the result of both a curved dilatational path intrinsic to CBR and a tissue region with dynamic anisotropic properties. The curved path places a high amount of compressive force on the tracheal rings, particularly on the adjacent superior tracheal ring, when the device orientation changes from a perpendicular to an inferior/caudad direction along the coaxial dilatational path that is at a maximum approximately half-way along the length of total dilation. Circular, concave dilator surfaces, such as in CBR, may also produce a more localized force distribution due to a single point of contact of the dilator on the tracheal rings. Specifically, the dilator places extreme pressure on the most midline and superficial portion of the adjacent superior ring to the tracheostomy being formed, typically on either the first or second tracheal ring of the trachea. This extreme force transmission results in excess anterior wall compression that often produces a tracheal ring fracture.

Arguably, tracheal ring fracture may lead to the development of granulation tissue that obstructs the airway, a condition known clinically as subglottic suprastomal stenosis, and may be the most important modifiable complication of PDT. (Epstein, 2005) Specifically, inappropriately high, non-uniformly distributed, and a non-uniform dilation force throughout the procedure, as in CBR, is thought to result in anterior tracheal wall compression and/or ring fracture further resulting in subglottic suprastomal stenosis. This subglottic suprastomal stenosis is fundamentally different from subglottic substomal stenosis as seen in surgical tracheostomy or irritation by a tracheostomy tube cuff or the distal edge of a tracheostomy tube. (Raghuraman, 2005) PDT subglottic stenosis is typically displayed sooner and located in the suprastomal region, i.e. the region above the dilatational ostomy/stoma, which typically has a smaller diameter, around 17 mm compared to 25 mm, and thus may be more prone to negative clinical outcomes. (Raghuraman, 2005)

The most creditable theory of the pathophysiology of subglottic stenosis involves the co-development of posterior tracheal wall laceration with tracheal ring fracture, destruction, accidental dislodgement, and/or surgical removal of the anterior/lateral tracheal ring, and/or anterior tracheal wall compression or impaction on the posterior wall. (Epstein, 2005) Furthermore, even without tracheal ring fracture, occurrences of excessive compression of the tracheal walls, isolated granulation, and/or pathologies producing hyperelastic tracheas as in patients with tracheomalacia or children, PDT patients can still develop subglottic suprastomal stenosis. (Dollner, 2002)

Several inventors have continued to introduce small changes to the CBR design, including, dilator lubrication via hydrophilic coating and dilator furrows or ridges that reduce insertion forces distally and increase gripping proximally. Several ergonomic enhancements have also been proposed for dilator devices with emphasis mainly on the handling of the device by the end-user. One such device developed and commercialized by Portex and called the Ultraperc or Uniperc (UP), utilizes a similar design as the CBR, but incorporates an ‘S’ Shaped curved handle at the proximal end (USPTO Application # US2004/0087991). Although this adds the possible benefit to allow for greater handling and to improve the standing posture of the provider while performing PDT, such improvements do little to reduce the risks of traumatic injury to the patient that can result directly from the shape and dilatational profile of the device.

The lack of modification(s), and adoption of devices thereof, to the fundamental shape and dilatational profile of PDT dilators may be due to the sophisticated, complex, and patient-specific nature of the neck anatomy. Of specific interest to the modification of PDT dilator profiles, several studies have shown that there exists two independent fiber families in the tracheal smooth muscle with dominance in the longitudinal direction in comparison to the circumferential direction, resulting in an increase of stiffness in the longitudinal direction as compared to the transverse. (Codd, Lambert, Alley, & Pack, 1994; Sarma, Pidaparti, & Meiss, 2002)

One proposed design to alter the dilatational profile of the single-pass dilator, the Ambesh T-Trach Dilator (ATD) invented by Dr. Sushil Ambesh, utilizes a transverse-biased ovaloid cross-sectional profile instead of a circular profile. By utilizing this ovaloid profile the insertion forces and level of compression are likely decreased due to the anisotropic properties of the tracheal smooth muscle that result in decreased stiffness in the transverse axis. (Ambesh, Tripathi, Pandey, Pant, & Singh, 2005) Reduced insertion forces results in decreased procedural time and further reduces the increase in peak airway pressure observed during PT procedures in general, which may be advantageous in patients who require higher inspired oxygen concentrations and/or have decreased lung compliance. (Ambesh et al., 2005)

Although, the constant oval cross-sectional profile of the ATD is a drawback to its design, as it may lead to difficulty producing an accurate sized stoma to insert most commonly used trachesostomy tube products that bear clearance profiles with a circular cross-section. This dilation inevitably results in insufficiently dilated half-ovaloid cross-sectional areas on the superior and inferior edges of the tracheal smooth muscle, cartilaginous rings, and pretracheal fascia in addition to unnecessary overdilated areas transverse to the ostomy. After dilating with the ATD the tube may become affixed in the pretracheal fascia creating a transferred compressive force to the trachea. This increased compressive force can result in tracheal ring fracture upon eventual access of the airway as well as posterior wall perforation immediately after passing the tracheal rings due to sudden loss of resistance.

Furthermore, when using a transverse-biased ovaloid dilation, it may be required for the transverse diameter to be increased by a certain factor to allow for sufficient dilation. Too much overdilation of the tracheal smooth muscle results in a higher risk of accidental decannulation and may produce tracheal wall damage if the transverse diameter of the dilator is too large for the patient's trachea. Conversely, too little overdilation may result in higher risk of tracheal damage as discussed above when introducing the tracheostomy tube. The overdilation of the ATD technique may not be applicable to smaller patients who cannot sustain such an increase in transverse diameter and may not be applicable to operators without the procedural experience necessary to understand the proper “ostomy” size and geometry to optimize procedural results. Specifically, roughly 15% of the female population and 2% of the male population has a transverse tracheal diameter of less then 14 mm, near the maximum diameter of dilation on the CBR. (Breatnach, Abbott, & Fraser, 1984; Griscom & Wohl, 1986) These rare patients may not be able to tolerate wholly transverse-biased dilation devices such as the ATD.

Another important factor in the adoption of PDT dilators has been the appropriate management of potential risk factors. The PDT products and/or techniques most adopted have consistently been the simplest designs involving specific variations of a key parameter/feature based on a critical analysis of existing literature to improve practicability and safety. (Byhahn et al., 2000) Thus, maintaining key procedural practices currently in place is necessary to improve minimally invasive dilatational procedure without significantly increasing their complexity, manufacturing constraints, risk of complications, and perceived risk from the creation of new iatrogenic risk factors.

Although CBR-like, single-pass continuously tapered curved, dilators currently produced for performing PDT are relatively easy to use and inexpensive to manufacture, they are not without their drawbacks. Clinically, there remain tremendous risks of unintended trauma to the patient receiving these interventions ranging from minor short term complications to severe and potentially costly long-term complications and these risks arise from a low level of specialized design specificity for procedural effectiveness as well as end-user ergonomics as discussed prior.

In the past, manufacturing methods such as plastics extruding of a single and/or multiple lumen tube followed by a heat forming process were used to create simple cross-sectional geometries, or dilatational profiles, of pre-defined curvatures using medical grade materials to form dilating devices for use in percutaneous dilatational procedures. As it relates to the production of single-pass dilators for use in performing PDT, these issues result in an inferiorly specified product that is ill suited for insertion into the specific tissue types encountered during this procedure. Additionally, the past manufacturing methods do not allow for the generation of clinically relevant device modifications for the unique anatomy of specific patients. Thus, these simple devices continue to cause traumatic injury to patients and present undo risk for physicians to adopt this clinically effective and tremendously cost-beneficial procedure.

Thus, the continuously tapered curved dilator devices that exist in the current state of the art in the field of the preferred embodiment, percutaneous dilatational tracheostomy dilator profile, suffer from a number of disadvantages:

(a) Their use results in the generation of both compressive and torque dilation forces on the trachea, specifically on the adjacent superior tracheal ring, caused by their generally curved dilation profiles that bear a continuous, increasing taper;

(b) The geometric profile of the curved tapered dilators commonly used in PDT results in the generation of, relatively, high dilatational force. This high level of force often occurs while the dilator is separating the pretracheal fascia due to the large cross-sectional profiles of the portion of the curved dilator in contact with the pretracheal fascia proximal to the portion of the dilator in contact with the tracheal smooth muscle and cartilaginous rings. This force is transferred to the cartilaginous rings of the trachea, specifically the adjacent superior tracheal ring, and induces tracheal ring fractures that may lead to subsequent complications including tracheal stenosis;

(c) The simple tapered circular geometry of the cross-sectional profile of PDT dilators fail to separate the adjacent stomal tracheal rings by transverse dilation prior to inducing the previously stated high dilatational force on the pretracheal fascia. This failure to perform transverse dilation prior to dilating in the longitudinal direction may specifically lead to subglottic suprastomal tracheal stenosis arising as a consequence of placement of a force capable of fracturing the adjacent superior tracheal ring;

(d) The isotropic or circularly symmetric cross-sectional dilatational profile of PDT dilators results in the generation of a non-uniform dilation force while passing through the varying layers of tissue impacted by tracheostomy producing a higher level of stress in the longitudinal, i.e. superior-inferior, direction of the cartilaginous ring and tracheal smooth muscle tissues.

(e) A generally convex, i.e. curved or circular, geometric profile on the superior and inferior dilatational profiles of PDT dilators cause a non-uniform distribution of force, specifically compressive force, upon the curved surface of the adjacent superior cartilaginous tracheal ring. This compressive force may cause tracheal ring fracture on the most anterior-medial aspect of the ring leading potentially to subsequent complications, including tracheal stenosis; and

(f) Posterior wall laceration and perforation caused by guidewire/guiding catheter kinking on the posterior wall (Byhahn et al., 2000) and/or misorientation of the guiding catheter to the dilator. (Trottier, 1999).

### REFERENCES

Ambesh, S., Tripathi, M., Pandey, C., Pant, K., & Singh, P. (2005). Clinical evaluation of the “T□Dagger™”: a new bedside percutaneous dilational tracheostomy device. Anaesthesia, 60, 708-711.
Beiderlinden, M., Karl Walz, M., Sander, A., Groeben, H., & Peters, J. X. R. (2002). Complications of bronchoscopically guided percutaneous dilational tracheostomy: beyond the learning curve. Intensive Care Medicine, 28(1), 59-62. doi:10.1007/s00134-001-1151-z
Breatnach, E., Abbott, G., & Fraser, R. (1984). Dimensions of the Normal Human Trachea. American Journal of Roentgenology, 142(5), 903-906.
Byhahn, C., Wilke, H., Halbig, S., Lischke, V., & Westphal, K. (2000). Percutaneous tracheostomy: Ciaglia Blue Rhino versus the basic ciaglia technique of percutaneous dilational tracheostomy. Anesthesia and Analgesia, 91(4), 882-886.
Codd, S., Lambert, R., Alley, M., & Pack, R. (1994). Tensile stiffness of ovine tracheal wall. The American Physiological Society.
Dollner, R. (2002). Laryngotracheoscopic Findings in Long-term Follow-up After Griggs Tracheostomy. Chest, 122(1), 206-212. doi:10.1378/chest. 122.1.206
Epstein, S. (2005). Late complications of tracheotomy Respiratory Care, 50(4).
Griscom, N., & Wohl, M. (1986). Dimensions of the Growing Trachea Related to Age and Gender. American Journal of Roentgenology, 146(2), 233-237.
Ho, E., Kapila, A., & Colquhoun-Flannery, W. (2005). Tracheal ring fracture and early tracheomalacia following percutaneous dilatational tracheostomy. BMC Ear, Nose and Throat Disorders, 5(1), 6. doi:10.1186/1472-6815-5-6
Raghuraman, G. (2005). Is Tracheal Stenosis Caused by Percutaneous Tracheostomy Different From That by Surgical Tracheostomy Chest, 127(3), 879-885. doi:10.1378/chest.127.3.879
S M Edwards, J. C. W. (2001). Tracheal cartilage fracture with the Blue Rhino Ciaglia percutaneous tracheostomy system. European Academy of Anaesthesiology, 1-1.
Sarma, P., Pidaparti, R., & Meiss, R. (2002). Anisotropic properties of tracheal smooth muscle tissue. Wiley Periodicals.
Thomas, A., Subramani, S., & Mitra, S. (2003). Tracheal ring fracture—dislodgement after Blue Rhino percutaneous tracheostomy. Anaesthesia Correspondence, 58, 1235-1252.
Trottier, S. J. (1999). Posterior Tracheal Wall Perforation During Percutaneous Dilational Tracheostomy: An Investigation Into Its Mechanism and Prevention. Chest, 115(5), 1383-1389. doi:10.1378/chest.115.5.1383

### BRIEF SUMMARY OF THE INVENTION

The present invention relates to a dilator device with a nonuniform taper based on the relevant anatomy to its specified surgical procedure. The dynamic geometric shape of the dilator is optimized through experimental and virtual testing to reduce, uniformly distribute, and ensure uniform, or constant temporal derivative of, dilatational force throughout dilation all serving to reduce trauma to tissues of the body that may result from dilation.

The device is comprised of several functionally specific portions that facilitate safe and proper use during a percutaneous dilatational procedure. There is a dilator on the distal portion of the device that bears the non-uniform dilatational profile that has been optimized to reduce trauma to the tissues of the body, a straight or curved handle on the proximal end of the device in distal continuity with the dilator for safe handling and for gaining leverage while inserting the dilator, a continuous generally circular channel that is integrated throughout the length of the device from the distal tip to the proximal end that is open on both ends for passage of a guidewire and/or catheter through the device, and a generally circular catheter, preferably made of Polytetrafluoroethylene (PTFE), arranged within the continuous channel, that is used to lower the risk of polymer scraping, friction, and kinking of the internal guidewire used in this procedure and which is known to those skilled in the art of the Seldinger Procedure.

The outward normal surface of the volume of the dilator, otherwise referred to as the geometric profile of the dilator, is defined by a series of parameters, it contains at least first order continuity at all points and it varies from its distal to its proximal ends continuously through multiple cross-sectional geometries. A dilator defining line defines the general shape of the dilator in a longitudinal cross-section. The centroids of the distinct cross-sectional surfaces are constrained to lie on a dilator centerline that may be offset from the line defining the shape of the dilator resulting in preferential biasing of specific dilator surface(s) to optimize procedural outcome, such as preferentially maintaining a flat or curved surface.

The resulting non-uniform, or asymmetric device profile of the present invention is optimized to allow for functionally-specific natural guidance of the medical instrument into the anatomy of interest with minimal complications and is particularly well suited for use in tracheostomy. Using analytical modeling, this asymmetric dilatational device profile can be optimized for use in a percutaneous dilatational procedure using parameter variations based on the relevant anatomy of the specific procedure and/or patient to result in a reduction of, uniform distribution of, and continuity of dilation force throughout the procedure. The process can be said to mimic the diversity of solenoglyphous (hollow) fangs that have naturally evolved across the Viperidae family of snakes with specificity for penetrating into the tissue of each particular species' natural prey. Thus, the generalization of the constraining geometry to include any number of asymmetric cross-sections and non-uniform segments to the defining dilator line allows the invention to become functionally-specific to a specified use and to optimize procedural outcomes.

An ensuing description with accompanying figures for one embodiment of the invention will further describe the advantages and application of this device.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “longitudinal” refers to the sagittal/superior-to-inferior axis/direction of a patient.

As used herein, the term “transverse” refers to the lateral/coronal axis/direction of a patient.

As used herein, the term “superior profile” refers to the surface of the dilator that contacts the superior tracheal ring and the posterior tracheal wall during PDT.

As used herein, the term “inferior profile” refers to the surface of the dilator that contacts the inferior tracheal ring and the anterior tracheal wall during PDT.

As used herein, the term “proximal” refers to a location on the device towards the handle.

As used herein, the term “distal” refers to a location on the device towards cross-section (i).

As used herein, the term “pretracheal fascia” refers to all fascial, musculature, neurovasculature, and skin components superficial to the trachea including: the skin, superficial cervical fascia, and the superficial and middle/visceral/pretracheal/tracheal portions of the deep cervical fascia.

As used herein, the term “PDT” refers to the percutaneous dilatational tracheostomy procedure.

As used herein, the term “Seldinger Procedure” refers to a method for performing percutaneous procedures including insertion of needle, identification of proper lumen location using negative pressure aspiration with a syringe and/or imaging, placement of a guidewire through the needle and into the lumen, and subsequent dilation over the wire of the intended structures.

As used herein, the term “dilation or dilatational force” refers to the amount of force required to insert a dilator to the required dilator cross-section necessary for tube insertion. The term “uniform dilation or dilatational force” refers to a roughly uniform force of dilation at each point in time throughout the procedure. The term “uniformly distributed dilation or dilatational force” refers to the distribution of the transferred dilatational force across the volume of the specific tissues of interest in the body, specifically a uniform distribution of force along the length of the adjacent superior tracheal ring.

As used herein, the term “convex” refers to a surface with an outward protrusion or an inward, negative, surface normal.

As used herein, the term “concave” refers to a surface with an inward indentation or an outward, positive, surface normal.

As used herein, the term “dilator/dilation/dilatational profile” refers to the outward normal surface of a volume defined by the parameters disclosed within containing at least first order continuity at all points.

As used herein, the term “taper length” refers to the length from cross-section (i), the distal end of the dilator, to cross-section (iv).

As used herein, the term “dilator defining line” refers to a line with three or more various segments that define the general path that the dilator profile follows from distal to proximal.

As used herein, the term “defining centerline” refers to a line offset from the dilator defining line such that there is a uniform anterior radius along the dilator from the dilator defining line of which the centroids of the five or more distinct cross-sectional surfaces are constrained to lie on.

As used herein, the term “centroid” refers to the center of mass of the cross-sectional surfaces.

As used herein, the term “cross-section (i)” refers to the most distal defining cross-section corresponding to a roughly circular thin walled surface.

As used herein, the term “cross-section (ii)” refers to the next proximal defining cross-section corresponding to a roughly transverse-biased ovaloid.

As used herein, the term “cross-section (iii)” refers to the next proximal defining cross-section corresponding to a roughly longitudinal-biased ovaloid.

As used herein, the term “cross-section (iv)” refers to the next proximal defining cross-section corresponding to a roughly circular or ovaloid surface,

As used herein, the term “cross-section (v)” refers to the most proximal defining cross-section corresponding to a roughly circular or ovaloid surface.

As used herein, the term “first order continuity” refers to continuity of the dilator surface to the first spatial derivative in three-dimensional space, i.e. G1 or C1 continuity to those skilled in the art.

As used herein, the term “CBR” refers to the Cook Medical Ciaglia Blue Rhino device and technique.

As used herein, the term “CBP” refers to the Cook Medical Ciaglia Basic Percutaneous device and technique.

As used herein, the term “UP” refers to the Smith's Medical Ultraperc or Uniperc device and technique.

As used herein, the term “ATD” refers to the Ambesh T-Dagger device and technique.

### BRIEF DESCRIPTION OF THE DRAWINGS

The invention will be described in conjunction with the following drawings in which like reference numerals designate like elements and wherein:

FIG. 1 is prior art of the existing standard of care for performance of PDT, the Cook Blue Rhino. (Cook Incorporated, Sabin Corporation, 2000) The device features a constant taper that dilates from a distal end to proximal end along a curve. The cross-section is circular throughout and the wall thickness is thinner at the distal end;

FIG. 2 is a side view, longitudinal or sagittal, drawing of an anatomical trachea model used for virtual and/or computational optimization of the device parameters;

FIG. 3 is a longitudinal device cross-section of the preferred embodiment with inner channel;

FIG. 4A is a side view drawing of the preferred embodiment;

FIG. 4B is a bottom view drawing of the preferred embodiment;

FIG. 4C is a back view drawing of the preferred embodiment;

FIG. 4D is a front view drawing of the preferred embodiment;

FIG. 5 is an off-set view drawing of the preferred embodiment; and,

FIG. 6 is a side view drawing of the preferred embodiment with defining device cross-sections expanded.

### DETAILED DESCRIPTION OF THE INVENTION

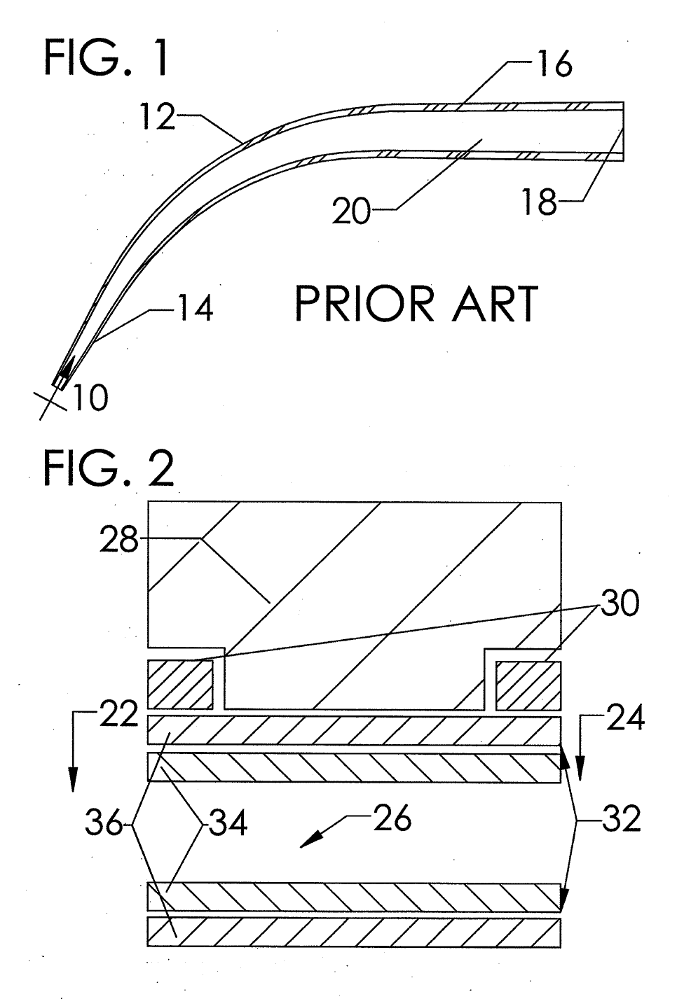

The dilator profile 10 of FIG. 1 is a cross-sectional profile of a prior art dilator first used in the “Ciaglia Blue Rhino Percutaneous Tracheostomy Introducer Set” that was introduced and sold by Cook Inc. for percutaneous dilatational tracheostomy with a single constantly tapered dilator. Dilator profile 10 bears a constant tapered surface profile extending from a curved distal portion 12 beginning from a distal tip portion 14, and extends continuously into a generally straight portion 16 that extends toward the proximal end 18. A central passageway 20 is shown extending through the device from distal tip portion 14 to proximal end 18.

An anatomical model of the trachea used for understanding and optimizing the parameters of the dilatational profile is shown in FIG. 2. Specified by the model are the regions of the anatomy superior to the tracheostomy 22 and inferior to the tracheostomy 24 that is formed into the lumen of the trachea 26. The trachea lies below a layer of pretracheal fascia 28. A percutaneous tracheostomy is formed between adjacent cartilaginous tracheal rings 30 by dilating the tracheal smooth muscle 32 to a sufficient size to place a tracheostomy tube into the lumen of the trachea 26. The tracheal smooth muscle 32 is composed of a longitudinal fiber family 34 and a circumferential fiber family 36 that contribute to the dynamic mechanical properties of the trachea as a whole.

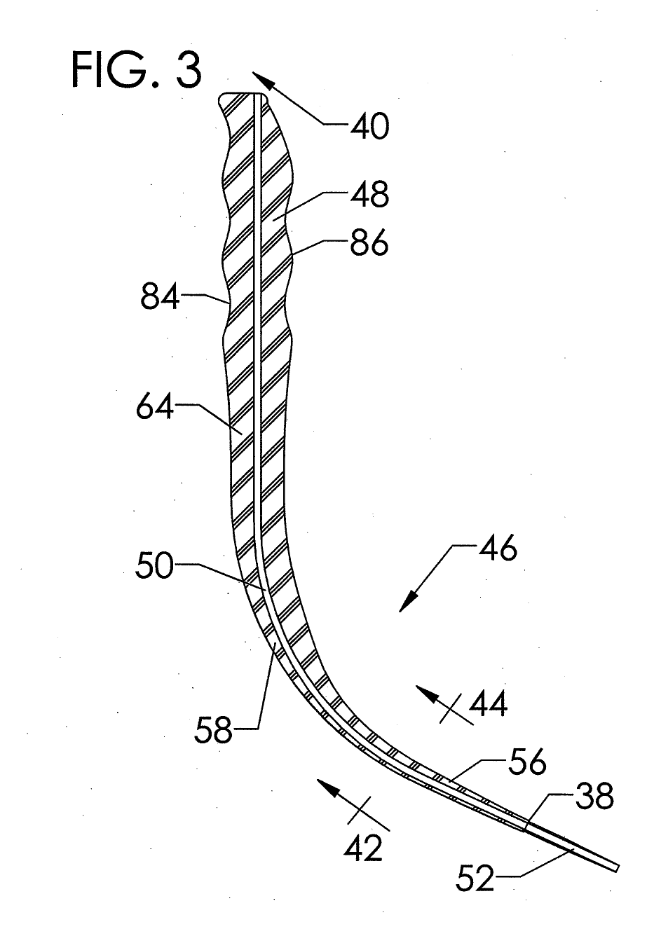

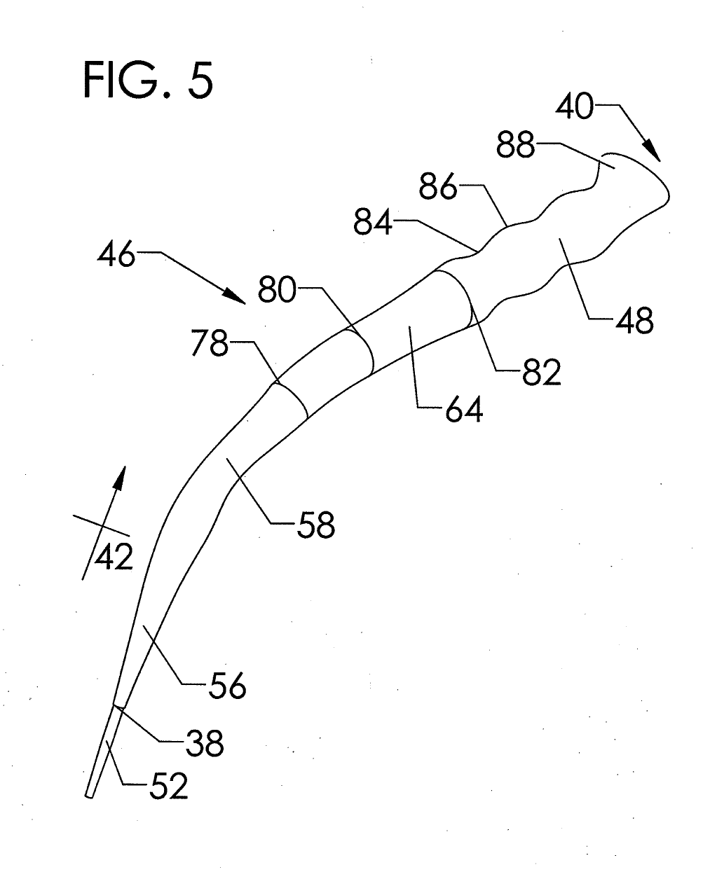

A preferred embodiment of the medical device of the present invention for performing a percutaneous dilatational tracheostomy (PDT) procedure is shown in FIGS. 3-6.

FIG. 3 shows the cross-sectional profile of the preferred embodiment demonstrating a dynamic and asymmetric tapered outer surface extending from a thin-walled distal tip 38 to a thick proximal end 40 through a series of non-uniform transitional stages.

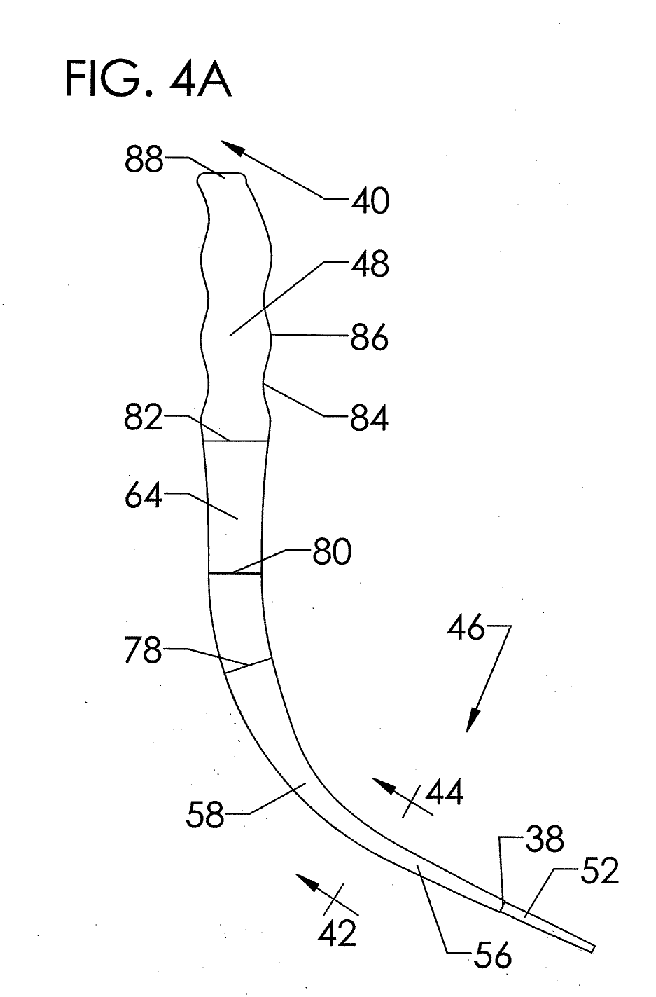

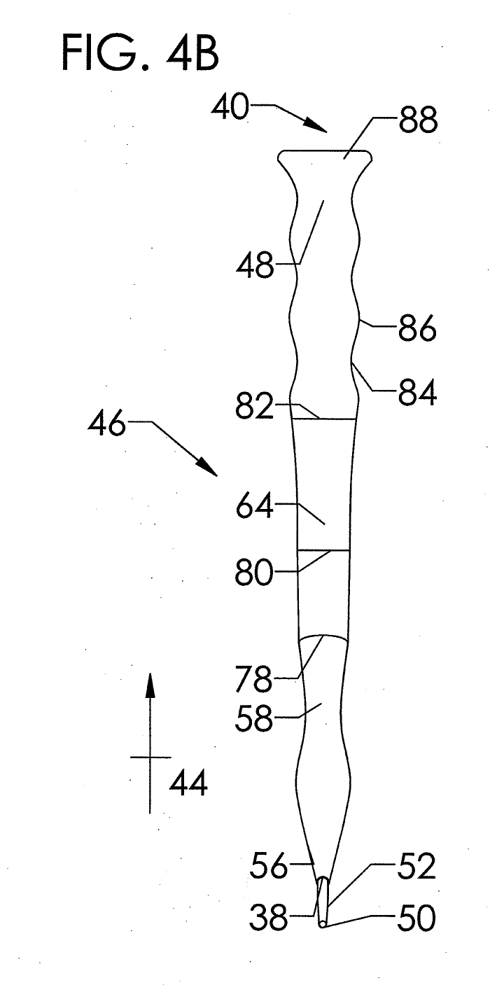

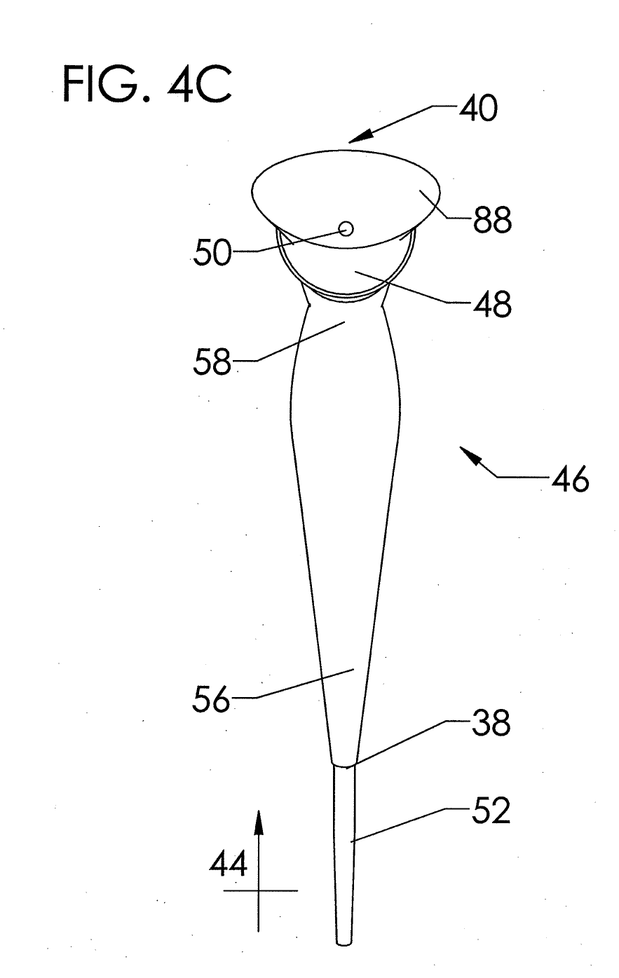

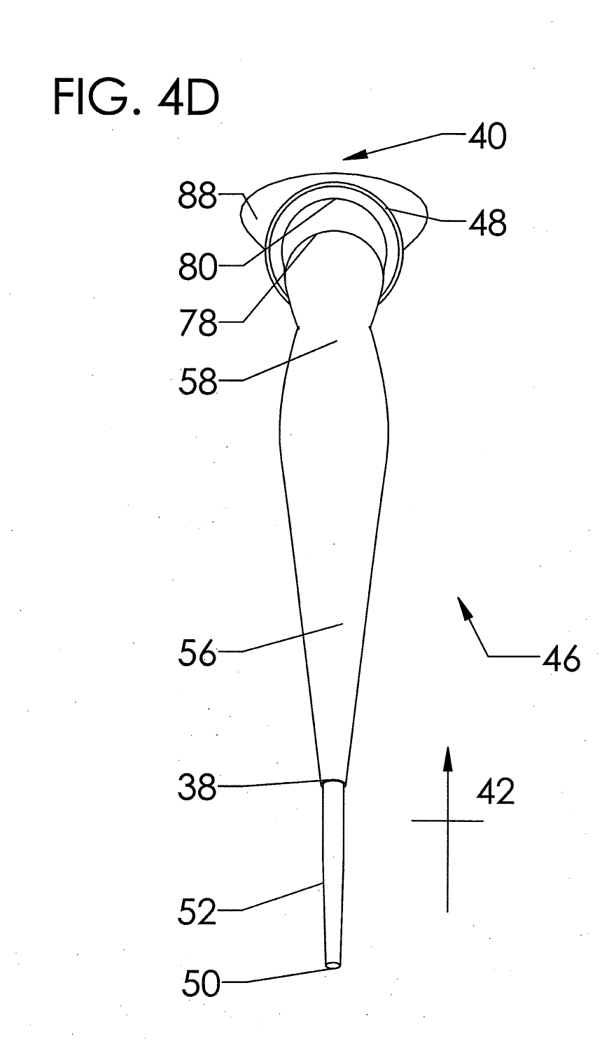

As shown in FIGS. 4A-D the device is comprised of a dilator 46 on the distal portion of the device, a straight or curved handle 48 on the proximal end 40 of the device in distal continuity with the dilator 46, a continuous generally circular channel 50 that is integrated throughout the length of the device from the distal tip 38 to the proximal end 40 that is open on both ends for passage of a guidewire and/or catheter through the device known to those skilled in the art of the Seldinger Procedure for medical interventions, and a generally circular catheter 52, preferably made of Polytetrafluoroethylene (PTFE), arranged within the continuous channel 50 via an overmold process known to those skilled in the art, or as a separate component that can be inserted into the channel, that is used to lower the risk of polymer scraping, friction, and kinking of the internal guidewire used in this procedure and which is known to those skilled in the art of the Seldinger Procedure.

The general shape of the dilator 46 is defined by a defining dilator line 54 segmented into three (3) sections along its length, including: a distal straight section 56 of length in range from 20-40 mm (preferred value=30 mm) that is proximally abutted by a curved section 58 defined by an angle of curvature 60 in range from 55-75□ (preferred value=65□) and a radius of curvature 62 in range from 70-90 mm (preferred value=80 mm) with a non-uniform, or asymmetric, profile that is proximally abutted by a straight extended length section 64 having length in range from 10-30 mm (preferred value=20 mm) between the location of sufficient intra-tracheal dilation at cross-section (iv) 72 and the proximal end of the dilator of roughly continuous cross-section throughout, which is comparable to the distal end of a tracheostomy tube, being generally circular or ovaloid in shape.

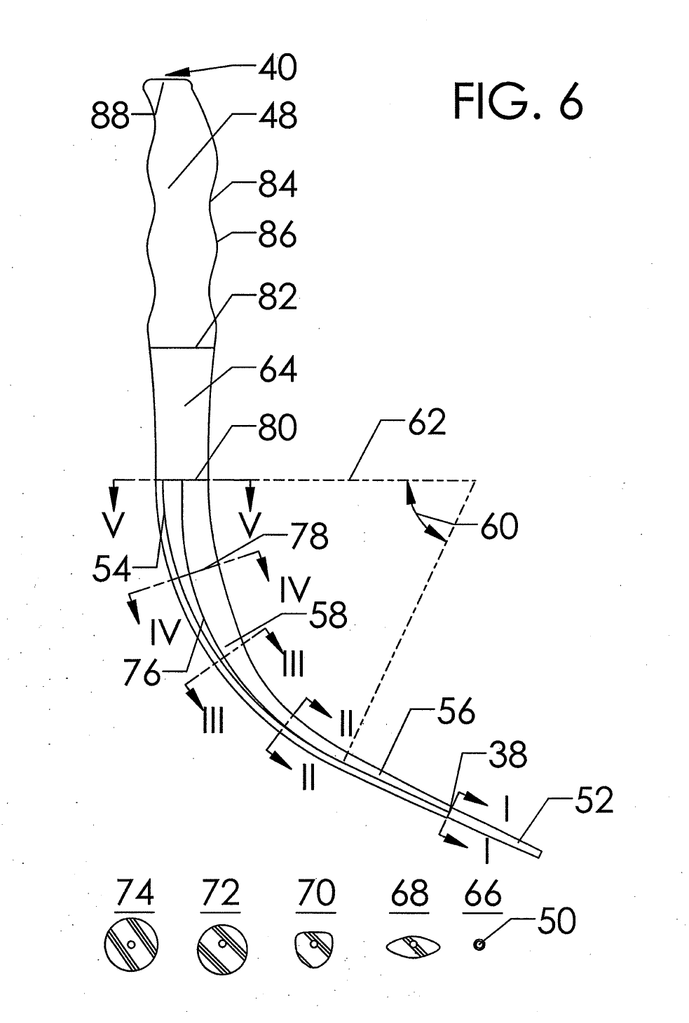

The geometric profile of the dilator 46, defined as the outward normal surface of its volume, contains at least first order continuity at all points and varies from its distal to its proximal ends continuously through five (5) distinct cross-sections, labeled (i) to (v) shown in FIG. 6. The centroids of the five distinct cross-sectional surfaces are constrained to lie on, and perpendicular to a dilator centerline 76, which is defined by a uniform anterior radius in range from 1-5 mm (preferred value=3.6 mm) from the defining dilator line 54 resulting in preferential biasing of the superior surface 42 of the dilator 46 to maintain a flat, or a broadly convex or concave, surface along its curvature.

Cross-section (i) 66 is located at the distal tip 38 of the dilator 46 and is a generally circular surface with a radius in range from 1-4 mm (preferred value=1.587 mm) and having wall thickness in a range from 0.1-1 mm (preferred value=0.254 mm) with smooth transition from the overmolded, or internally placed, catheter 52. From cross-section (i) 66 the proximal cross-sections of the dilator 46 gradually expand from distal to proximal in a predominately transverse-biased taper to abut cross-section (ii) 68 located proximally.

Cross-section (ii) 68 is a roughly transverse-biased ovaloid surface located in a range 25-75%, with a preferred value of 50%, along the total tapered length of the dilator 46, defined as the length from cross-section (i) 66 to cross-section (iv) 72, as measured from the distal end of the dilator 46, and having a maximal transverse diameter in range from 10-15 mm (preferred value=13.5 mm). Cross-section (ii) 68 has a longitudinal diameter in range from 25-75% (preferred value=50%), of a defined continuously increasing taper from cross-section (i) 66 to cross-section (iv) 72. From cross-section (ii) 68, the surface of the dilator 46 has an approximately flat, or slightly concave or convex, superior dilatational profile sufficient for uniformly distributing force transversely across the 22 tracheal ring and a triangularly filleted convexity on the inferior dilatational profile 40 to reduce surface area and frictional/resistive forces on the tracheal smooth muscle 32, cartilaginous rings, and/or pretracheal fascia 28. Also, from cross-section (ii) 68 the proximal cross-sections gradually change from distal to proximal expanding predominately in a longitudinal-biased taper while contracting in the transverse direction to abut cross-section (iii) 70 located proximally.

Cross-section (iii) 70 has a generally longitudinal-biased ovaloid surface located in range from 50-95% (preferred value=75%) along the total taper length of the dilator 46 from the distal tip 38 of the device. Cross-section (iii) 70 has a longitudinal diameter in range from 100-175% (preferred value=125%), of a defined continuously increasing taper from the longitudinal diameter at cross-section (ii) 68 to the longitudinal diameter of cross-section (iv) 72 and has a minimal transverse diameter in the range from 5-13.5 mm (preferred value=10.125 mm or corresponding to the minimal value sufficient for uniformly distributing force along the superior tracheal ring). From cross-section (iii) 70 the surface profile properties of the dilator 46 are in continuity with distal surface properties of cross-section (ii) 68 having an approximately flat, or slightly concave or convex, superior dilatational surface 42 profile and a triangularly filleted convexity on the inferior dilatational surface 44 profile to reduce surface area and frictional/resistive forces on the tracheal smooth muscle 32, cartilaginous rings, and/or pretracheal fascia 28. However, from cross-section (iii) 70 the proximal cross-sections gradually expand from distal to proximal in both transverse and longitudinal-biased continuous tapers to abut cross-section (iv) 72 located proximally.

Cross-section (iv) 72, located at the distal end of the straight extended length portion 64 of the dilator 46 has a geometrical shape analogous to a distal tracheostomy tube cross-section of roughly circular or ovaloid dimension with a roughly circular diameter in the range from 10-13.5 mm (preferred value=12.67 mm). The device bears a 38 Fr. Marking 78 at cross-section (iv) 72 to indicate the proper tracheal dilation stop point to the end user. From cross-section (iv) 72 the proximal cross-sections gradually expand from distal to proximal in generally equal transverse and longitudinal-biased tapers for a length in range of 5 to 45 mm (preferred value=25.6 mm) to abut cross-section (v) 74 located proximally.

Cross-section (v) 74 has a geometrical shape analogous to a distal tracheostomy tube cross-section of roughly circular or ovaloid dimension located at the proximal end of the straight extended length portion 64 of the dilator 46. Cross-section (v) 74 has a roughly circular diameter in range from 12.67-15 mm (preferred value=13.5 mm) to allow for adequate additional dilation of pretracheal fascia 28, tracheal dilation to a larger tube size, and/or dilation in an obese patient having a greater than average pretracheal distance. The device bears a skin-level marking 80 at cross-section (v) indicating the maximum insertion point. From cross-section (v) 74 the proximal cross-sections maintain a relatively similar shape and diameter along the proximal continuity of the dilator until abutting proximally to the straight, or curved, handle 48.

The straight, or curved, handle 48 on the proximal end 40 of the device is arranged to be contiguous to the dilator 46 having distal continuity with the proximal end of the dilator 46 and is comprised of; a distal circular cross-section 82 of continuity to the proximal end of the dilator 46 which gradually outwardly tapers along a length of range from 5-25 mm (preferred value=20 mm), a series of concave 84 and convex 86 profiles numbered in range from 2-5 (preferred value=3 concave and 4 convex continuous profiles of length roughly 10 mm from convexity to concavity and starting and ending on said convex profiles) for a hand grip, and a proximal end consisting of a T-grip 88, or symmetric transverse outcroppings, for abutting to the plantar component of the providers hand.

The intended procedural use of the device of the preferred embodiment is with the common Seldinger Technique used in surgical procedures with single-dilator PDT sets and known to those skilled in the art.

The benefits of this invention arise from the non-uniform surface geometry constrained by the parameters of the varying cross-sectional profiles of the dilator portion that are chosen based on study of the relevant anatomy and optimized through experimental and virtual testing. Accordingly, use of this dynamic dilatational shape in PDT results in a reduction in dilatational force, a uniform distribution of force, specifically along the adjacent superior tracheal ring as the device is passed through the tissue layers above and into the lumen of trachea, and it ensures a uniform, or constant temporal derivative of, dilatational force all throughout the period of dilation during the procedure. These benefits have the potential generally to reduce trauma to tissues of the body resulting from dilation, and specifically to yield a reduction in anterior tracheal wall compression, posterior wall laceration/perforation, and tracheal ring fracture which may subsequently lead to subglottic suprastomal stenosis. Furthermore the present invention provides these benefits specifically in that:

a. It performs initial dilation across the transverse aspect of the tracheal smooth muscle with minimal longitudinal diameter and minimal force/compression of the anterior tracheal wall to separate the smooth muscle fascia prior to dilating in the lateral direction because the tissue is known to be at least 3-fold less stiff in the transverse.
b. In distinction from current techniques that also initially dilate in the transverse direction, the dilator of the present invention tapers dynamically to a specific, marked generally circular cross-section that is known to create adequate dilation for subsequent tracheostomy tube insertion with limited tracheal compression.
c. The longitudinally thin-walled and transverse-biased distal dilator profile may not only minimize force/compression but also enhance mobility of the distal section of the device, which may further lower force applied to the posterior wall and risks of posterior wall perforation.
d. The generally flat, or concave or convex, surface on the anterior surface 42 of the dilator 46 extending from the distal tip 38 to cross-section (iii) 70 distributes force evenly across the adjacent superior ring: first or second, recognizable by a uniform anterior wall compression, reduced anterior wall compression, and reduced incidence of tracheal ring fracture.
e. The catheter 52 being integrated into the dilator 46 as one component will reduce the number of steps/complexity and may reduce risks of posterior wall perforation due to either guidewire/guiding catheter kinking on the posterior wall or misorientation of the guiding catheter to the dilator.
f. The device can be handled in a pencil-tip technique by gripping at the transverse indentation located at the center of the device, near cross-section (iii) 70 or in a full hand-grip fashion by gripping the finger protrusions 84, 86 on the handle 48 at the proximal end of the device respectively.
g. The non-uniform asymmetric taper along the length of dilator 46 will result in generation of a uniform dilation during the procedure ensure continuity of force production and distribution on the tracheal rings as compared to the continuous symmetric taper of CBR.
h. The procedure for using the device remains consistent with the Seldinger technique used by devices of the prior art to limit the learning curve for providers choosing to use this device, thus lowering the risk of iatrogenic complications related to using a new device.

In another embodiment, the invention relates to other parameter variations from the generalized preferred embodiment based on the relevant anatomy of the specific procedure and/or patient to result in a reduction of, uniform distribution of, and continuity of dilation force throughout the procedure.

In another embodiment, the invention relates to the generalization of the constraining geometry to include any number of cross-sections and segments to the defining dilator line to optimize procedural outcomes.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

What is claimed is:

1. A device for providing an airway through the pretracheal fascia and tracheal wall of a patient that reduces trauma to tracheal smooth muscle, cartilaginous rings, and/or pretracheal fascia, and/or adjacent anatomical structures, said device comprising:
a. a distal region having a generally circular cross-sectional profile and a tip;
b. an intermediate region located proximally to said distal region and having a dilatational surface profile including a cross-section shaped in the form of a generally transverse-biased ovaloid;
c. a proximal region located at the proximal end of said device and including a dilatational surface profile analogous to a tracheostomy tube; and,
d. a passageway extending through said device defined by a wall having a thickness, said passageway opening at said tip to accommodate a guide member.

2. The device of claim 1, additionally comprising a handle in abutment with said proximal region.

3. The device of claim 1, wherein said handle is straight.

4. The device of claim 1, wherein said handle is curved.

5. The device of claim 1, wherein said transverse-biased ovaloid surface includes a superior dilatational profile selected from the group consisting of generally flat, slightly concave, and slightly convex.

6. The device of claim 1, wherein said transverse-biased ovaloid surface includes a maximal transverse diameter in a range from 10 to 15 mm.

7. The device of claim 6, wherein said transverse-biased ovaloid surface includes a maximal transverse diameter of approximately 13.5 mm.

8. The device of claim 1, wherein the general shape of said device includes a distally located straight length, a proximally located straight length and an intermediate curved length located between said straight lengths, said intermediate curved length including an angle of curvature in a range from 55 to 75 degrees and a radius of curvature in a range of 70 to 90 mm.

9. The device of claim 8, wherein said angle of curvature is approximately 65 degrees and said radius of curvature is approximately 80 mm.

10. The device of claim 1, wherein said proximal region is of a generally circular cross-sectional profile.

11. The device of claim 2, wherein said handle includes a T-grip.

12. The device of claim 2, wherein said handle includes finger protrusions.

13. The device of claim 1, wherein said device includes a water-activated lubricious coating on the surface thereof.

14. The device of claim 1, additionally comprising a catheter disposed within said passageway and extending distally from said tip.

15. The device of claim 6, wherein said maximal transverse diameter is the maximal diameter of the device.

16. The device of claim 1, wherein the minimum diameter of the device is located at the tip, and wherein said tip includes a generally circular surface profile.

17. The device of claim 1, wherein the dilatational surface of the proximal region is sufficiently large to enable insertion of a tracheostomy tube through the pretracheal fascia and tracheal wall of the patient after removal of the device from the patient's trachea.

18. The device of claim 1, wherein the device includes a taper that extends in a non-uniform manner over the length of the device.

19. The device of claim 1, wherein said transverse-biased ovaloid surface includes a slightly concave superior dilatational profile.

20. The device of claim 1, additionally comprising a dilator defining line extending along the length of said device, said line defining a profile of said device from said distal region to said proximal region.

21. The device of claim 1, additionally comprising a region located between said intermediate and proximal regions, said between located region including a generally longitudinal-biased ovaloid dilatational surface profile.

22. A device for providing an airway through the pretracheal fascia and tracheal wall of a patient that reduces trauma to tracheal smooth muscle, cartilaginous rings, and/or pretracheal fascia, and/or adjacent anatomical structures, said device comprising:
a. a narrower distal region having a tip;
b. a wider proximal region, said device extending along a length from said narrower distal region to said wider proximal region, said length comprising a non-uniform surface geometry including varying cross-sectional profiles, said device generally tapering outwardly as it extends from said narrower distal region along said length to said wider proximal region; and,
c. a passageway extending through said device defined by a wall having a thickness, said passageway opening at said tip to accommodate a guide member.
